(19) United States
(12) Patent Application Publication
(10) Pub. No.: US 2016/0096030 A1
(43) Pub. Date: Apr. 7, 2016

(54) PULSED GRADIENT FIELD METHOD TO COUNTERACT A STATIC MAGNETIC FIELD FOR MAGNETIC PARTICLE FOCUSING

(71) Applicant: WEINBERG MEDICAL PHYSICS LLC, Bethesda, MD (US)

(72) Inventors: Aleksandar Nelson NACEV, Bethesda, MD (US); Irving N. WEINBERG, Bethesda, MD (US)

(73) Assignee: Weinberg Medical Physics LLC

(21) Appl. No.: 14/873,738

(22) Filed: Oct. 2, 2015

Related U.S. Application Data
(60) Provisional application No. 62/058,874, filed on Oct. 2, 2014.

(51) Int. Cl. A61N 2/00; A61B 5/055; G01R 33/48

(52) U.S. Cl. CPC A61N 2/002; G01R 33/48; A61B 5/055

(57) ABSTRACT

Disclosed embodiments provide an apparatus and method that produce a magnetic field gradient configured to counteract effects of a static magnetic field such that the combination of the two fields may be applied to one or more magnetic particles to manipulate the magnetic particle(s).

### CROSS REFERENCE

This application relies for priority on U.S. Provisional Application No. U.S. 62/058,874 filed Oct. 2, 2014 and entitled “PULSED GRADIENT FIELD METHOD TO COUNTERACT A STATIC MAGNETIC FIELD FOR MAGNETIC PARTICLE FOCUSING,” the contents of which are incorporated herein by reference.

### FIELD

Disclosed embodiments pertain to a technique and components for manipulating the location of one or more magnetizable particles. More specifically, the disclosed embodiments provide the ability to push or focus particles by the application of a magnetic gradient field.

### SUMMARY

Disclosed embodiments provide an apparatus and method that produce a magnetic field gradient configured to counteract effects of a static magnetic field such that the combination of the two fields may be applied to one or more magnetizable particles to manipulate the magnetizable particle(s).

In accordance with at least one disclosed embodiment, application of the magnetic fields is used to focus the magnetizable particles in a particular location, for example, within a body part.

### BRIEF DESCRIPTION OF THE FIGURES

A more complete understanding of the present invention and the utility thereof may be acquired by referring to the following description in consideration of the accompanying drawings, in which like reference numbers indicate like features, and wherein:

FIG. 1 illustrates one example of a methodology provided in accordance with the disclosed embodiments.

FIG. 2 represents a one-dimensional example of the invented apparatus used to implement the method shown in FIG. 1.

### DETAILED DESCRIPTION

As a result of one of the innovations of one of the named inventors, it is now possible to pulse magnetic fields to push magnetizable particles and focus them to spots at a location between magnetic sources. See, for example, US Pat. Pub. 20140309479, entitled “SYSTEM, METHOD AND EQUIPMENT FOR IMPLEMENTING TEMPORARY DIAMAGNETIC PROPULSIVE FOCUSING EFFECT WITH TRANSIENT APPLIED MAGNETIC FIELD PULSES” and filed on Feb. 18, 2014 (incorporated by reference in its entirety). The technique described in that application relied upon separate magnetic field pulses to first polarize the particles into a defined orientation, and then, subsequently, apply a gradient field aligned opposite to the polarizing field to push them away from the magnetic source. The principle of this method exploited the magnetic alignment of the magnetizable particle.

While that prior patent application used separate pulses for the polarizing and gradient magnetic fields, it is further possible to use a combination of static magnetic fields and pulsed magnetic fields to push and focus the magnetizable particles. Such is the subject of the present disclosure.

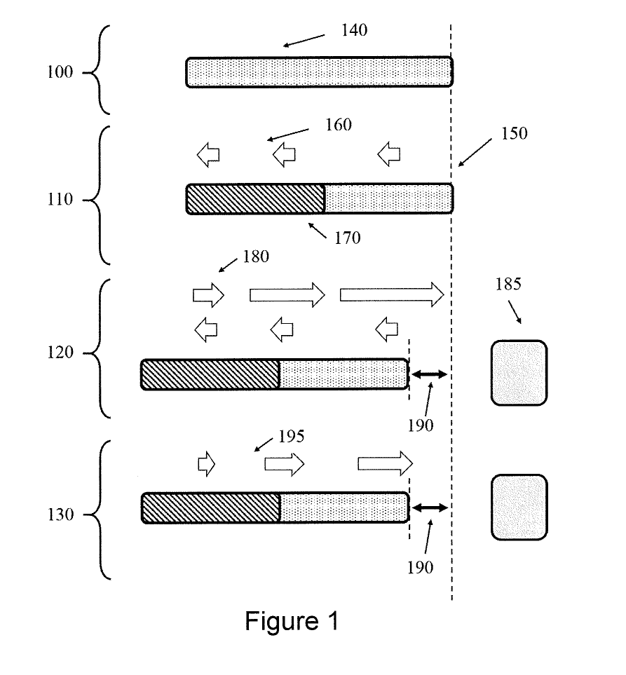

In order to achieve this functionality, the applied pulsed magnetic field must be strong enough to overcome the static magnetic field within a region of interest. Thus, as illustrated in FIG. 1, a methodology for applying a combination of static and pulsed magnetic fields is provided wherein the magnetizable particles can be pushed and focused within that region of interest.

FIG. 1 illustrates an embodiment for pushing a particle in one-dimension with a static polarizing field and a pulsed gradient field. As shown in FIG. 1, the methodology begins at 100, at which a magnetizable particle 140 starts at position 150. Subsequently, at 110, a static uniform magnetic field 160 is applied to the particle. This uniform magnetic field polarizes the particle and grants the particle some preferred particle alignment 170.

Subsequently, at time 120, a pulsed, gradient magnetic field 180 produced by a magnetic source 185 is additionally applied to the particle that is opposite in alignment from the polarizing field 160 and, therefore, the particle alignment 170 established at 110. Since the gradient magnetic field 180 is sufficiently strong compared to the polarizing field 160, the resultant total magnetic field 195 is oriented in the same direction as the pulsed gradient magnetic field 180 with only a smaller magnetic field intensity, as illustrated at 130. As a result, the resultant magnetic field would instead push the particle a distance 190 from the initial position 150 away from the magnetic source 185.

It should be understood that, by the linear properties of magnetic fields, the operations denoted at 120 and 130 are equivalent. The combination of magnetic fields generated in operation 120 produces an equivalent magnetic field as in operation 130.

Operations performed at 110, 120, and/or 130 can be repeated as many times and in as many directions as needed in order to achieve focusing of the magnetizable particle.

Alternative configurations of the resulting combined magnetic field are also possible. For example, a static gradient field and a pulsed uniform magnetic field. This is because the principle of magnetic field superposition still applies.

In addition, the uniform field need not be perfectly uniform; rather, it may include a built in gradient such that the movement 190 illustrated in FIG. 1 may be greater than any accrued displacement due to the uniform field.

In accordance with at least one embodiment, time-varying magnetic gradients may be applied to a region of space within which a static magnetic field is already present. In such an embodiment, a static magnetic field may be used to align a magnetizable particle within this region. Subsequently, the applied time-varying magnetic gradient field adds to the static magnetic field such that the resultant total magnetic field in the region of space may be a gradient field aligned opposite to the static magnetic field. Moreover, the resultant total magnetic field in the region of space may increase towards the magnetic source.

Thus, during the application of the time-varying magnetic field, the magnetized particle may be aligned opposite to the resultant magnetic field. Since the particle's alignment would, thus, be opposite to the field, the particle would experience a force pushing it away from the magnetic source. However, once the time-varying magnetic field is removed, the particle will again align to the static magnetic field.

After an amount of time necessary for the appropriate realignment of the magnetizable particle to the static magnetic field, the time-varying magnetic gradient field can be re-applied to again push the magetizable particle away from the magnetic source.

In accordance with at least one embodiment, application of magnetic fields creates a nodal point, a local minimum of magnetic field strength as is usually created by a magnetic field cancelation point, within the region of interest as published by A. Sarwar, A. Nemirovski, and B. Shapiro entitled “OPTIMAL HALBACH PERMANENT MAGNET DESIGNS FOR MAXIMALLY PULLING AND PUSHING NANOPARTICLES” in March 2012 in the Journal of Magnetism and Magnetic Materials (Volume 324, Issue 5) (incorporated by reference in its entirety). This nodal point would push magnetizable particles away from the nodal point in all directions and therefore as well as in a direction away from the magnetic source, but the application of such a nodal point would create magnetic forces that disperse the magnetizable particles instead of focusing them to a target location.

In accordance with at least one embodiment, the application of the at least one static magnetic field and the at least one time-varying magnetic field creates a dispersal region for at least one magnetizable particle. In accordance with the described apparatus, magnetic forces are capable of either focusing particles or dispersing magnetizable particles. It is possible, as with the creation of a magnetic field nodal point, that a magnetizable particle would be dispersed and pushed away from each other magnetizable particle. It is also possible, with the same apparatus and with subsequent magnetic field applications that the magnetizable particle can be focused to a region in proximity to the dispersal region.

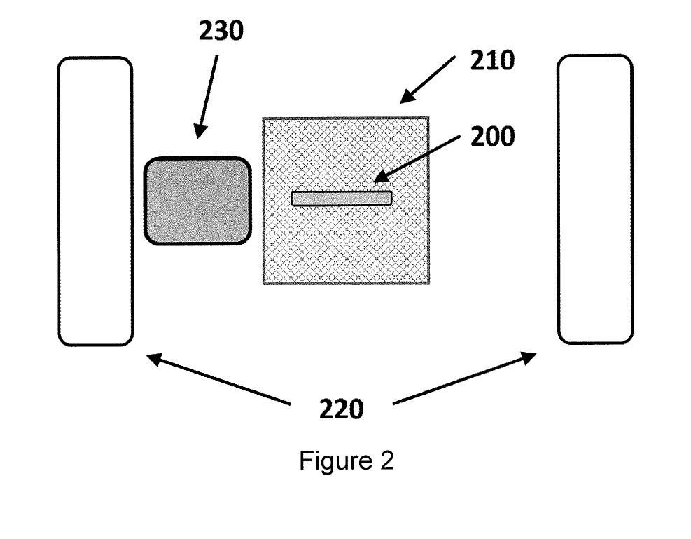

FIG. 2 illustrates one example of an apparatus that may be used for one dimensional movement in accordance with the disclosed embodiments. A magnetizable particle, 200, is located within a region of interest, 210. Within this region, a static magnetic field, 160 of FIG. 1, is created by one or more permanent magnets or electromagnets, 220. This static magnetic field may have a high degree of uniformity as would be the case proposed in FIG. 2, but high uniformity is not required. Lastly, a gradient magnetic field, 180 of FIG. 1, is applied by applying power through an electromagnet, 230, in proximity to the region of interest 210. This gradient magnetic field has the characteristics of canceling the static magnetic field produced by magnets 220 in the region of interest. This gradient magnetic field also produces a magnetic gradient which increases in magnitude in proximity to magnet 230. This gradient magnetic field is aligned in a direction that is opposed to the static magnetic field as shown in the method illustrated by FIG. 1. The gradient magnetic field is shown in this embodiment as produced by an electromagnet, but it is possible that magnet 230 is a permanent magnet that is instead physically oscillated in space around region 210 creating a time varying gradient field as described by the proposed method.

It should be understood that, although the above described embodiments have been explained in conjunction with a single dimension of movement, the disclosed embodiments are not limited to one dimensional movement. Rather, three other embodiments include using additional magnetic sources placed around the region of interest, physically rotating around the region of interest the magnetic source apparatus described previously in FIG. 2 so that the apparatus would operate in the other two spatial dimensions, or creating a magnetic source apparatus that can generate magnetic force fields that have a magnetic force gradient in each of the three spatial directions.

Application of magnetic fields as in FIG. 2 can be used to induce an anti-agglomeration behavior in a plurality of magnetizable particles including the at least one magnetizable particle. A plurality of magnetizable particles can be disassociated in their behavior by performing one or more of the following actions: An applied magnetic field can be rotated in direction to introduce a time varying torque acting upon the magnetic particle. An applied magnetic field can be changed in magnetic field intensity in combination with a static magnetic field so that the resultant magnetic field creates a time varying torque. An applied magnetic field can be changed in magnetic field intensity with or without a static magnetic field by which the resultant magnetic field creates a time varying magnetic force.

It should be understood that the components illustrated in FIG. 2 and their associated functionality explained in conjunction with FIG. 1, may be implemented in conjunction with, or under the control of, one or more general purpose computers running software algorithms to provide the presently disclosed functionality and turning those computers into specific purpose computers.

It is understood that although the apparatus of FIG. 2 can create a one-dimensional magnetic propulsive force, further embodiments include the addition of either electromagnets or permanent magnets designed to introduce time varying magnetic gradient fields in alternative directions. Electromagnets can but are not limited to producing time varying magnetic gradient fields by the changing of electric power flowing through the electromagnet. Further embodiments include moving a metallic core into and out of a solenoid electromagnet or permanent magnet, or by moving a metallic object near to an electromagnet or permanent magnet. Both electromagnets and permanent magnets can be moved and rotated in space to create a magnetic field that varies in time.

It is understood that at least one of the plurality of magnets used in FIG. 2 is at least one electromagnetic coil that is cooled to increase magnetic field strength thereof. The magnetic field produced by an electromagnet is directly proportional to the current flowing through the electromagnet windings. The current through this coil is limited by the power supplied to the coil. For a given electromagnet, the power is the mathematical product of the resistance of the electromagnet and the current flowing through the windings of the electromagnet. As electromagnets are composed primarily of conductive materials, the conductivity increases as the material temperature decreases. By cooling the electromagnets, the resistance of the electromagnet would decrease. Thus for the same power applied to the electromagnet, the current would be higher thereby creating a stronger magnetic field. It is important to note that not all materials begin conductive and are only conductive as their temperature decreases, as in the case of superconducting materials

It is understood in FIG. 2 that at least one of the plurality of magnets is a magnet assembly that is near to the region of interest or encompasses the region of interest. The magnet assembly may include permanent magnets, powered electromagnets, or energy bearing superconducting magnets. The magnetic fields generated by these apparatuses is localized to the apparatuses. Therefore, the region of interest requires at least one magnet assembly located near to the region of interest in order for a magnetic field to be applied within the region of interest.

The apparatus of FIG. 2, further comprising a field shifting apparatus comprised of ferromagnetic mu-metal materials and/or superconducting materials which alters a location of the at least one static magnetic field and the at least one time-varying magnetic field as published by J. Prat-Camps, C. Navau, and A. Sanchez as “A MAGNETIC WORMHOLE” in August 2015 Scientific Reports (incorporated by reference in its entirety). With the application of such an apparatus, the magnetic fields generated can be shaped in such a way as to affect the magnetic forces acting upon a magnetizable particles either by increasing, decreasing or reshaping the magnetic force field.

It is understood that the apparatus of FIG. 2 may comprise a metallic component which alters the shape and strength of the at least one static magnetic field and the at least one time-varying magnetic field. With the application of such an apparatus, the metal will concentrate and refocus the magnetic field thereby allowing for a shaping of the magnetic field within the region of interest. This reshaping can either increase or decrease the magnetic force field.

Moreover, those skilled in the art will recognize, upon consideration of the above teachings, that the above exemplary embodiments may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments of the present invention. Such alternative storage devices should be considered equivalents.

There are various applications for the focusing of particles, for example, nanoparticles within a human body part. For example, in a similar manner to that discussed in US Pat. Pub. 20130296631, entitled “CLEANING ARTERIOSCLEROTIC VESSELS WITH MAGNETIC NANOSWIMMERS,” filed May 7, 2013 (incorporated by reference in its entirety), presently disclosed embodiments may be used in an apparatus and method for brushing plaques from vessels by exposing magnetizable nanoparticles to changing magnetic gradients.

More specifically, as explained above, the disclosed embodiments may be utilized to implement multi-dimensional motion of a plurality of magnetizable particles. Accordingly, presently disclosed embodiments may be used to induce rotatory motion magnetizable particles, e.g., nanoparticles, to remove plaque material from plaque surfaces, wherein removed material can be subsequently removed through a catheter or alternatively using natural flow through the vessel. Thus, a configuration of magnetic elements (e.g., electromagnetic or permanent magnetic material) may be provided, configured and utilized to push (i.e., repel) or pull (i.e., attract) the magnetizable particles to specified locations within a patient's body, e.g., areas of vessels within the body identified via imaging (or other techniques) as suffering from plaque build-up.

Accordingly, it should be understood that the presently disclosed embodiments may be utilized to provide the functionality disclosed in US Pat. Pub. 20130296631.

Additionally, it should be understood that disclosed embodiments may be utilized by incorporating one or more propulsive coils into a Magnetic Resonance Imaging (MRI) scanning system (for example, to retrofit a conventional MRI scanning system; in such an implementation the propulsive coils and other hardware and software necessary to direct the magnetizable particles to the vessels of interest and/or implement the disclosed embodiment for removing plaque from blood vessel walls under direction of imaging completed by an MRI scanning system may be included in a kit for installation as part of such a retrofit or upgrade). Such a configuration was taught by the inventor in U.S. patent application Ser. No. 13/586,489 entitled “MRI-GUIDED NANOPARTICLE CANCER THERAPY APPARATUS AND METHODOLOGY” (and is incorporated by reference in its entirety).

Thus, the presently disclosed embodiments may be utilized to manipulate and agitate particles within an MRI system while a physician may visualize a concentration of nanoparticles within a body part in the course of their manipulation. In such an implementation, the magnetic gradients used to manipulate the particles are not contemporaneous with the pulsed magnetic gradients used by the MRI system to create an image of the body and/or particles in the body. For example, the propulsive magnetic gradient pulses are interleaved with the magnetic gradients used for imaging purposes, or may precede or follow the magnetic gradients used for imaging purposes. This lack of contemporaneity implies that the pulsed magnetic fields used to propel the nanoparticles do not interfere with the process of collecting an image with the MRI scanner, where “interference” is defined for the purposes of this description as a process that would cause reduced quality of the MRI scanner image.

In at least one alternative embodiment, strong pulsed magnetic gradients may be used to propel magnetizable particles and also as part of the process of creating an image of the body and/or magnetizable particles in a patient's body, wherein the magnetic gradients used to create an image are of low magnitude and magnetic gradients used to manipulate the location of particles employ features disclosed in U.S. Pat. No. 8,154,286, by one of the named inventors, entitled “APPARATUS AND METHOD FOR DECREASING BIO-EFFECTS OF MAGNETIC FIELDS”, issued Apr. 10, 2012 (and incorporated by reference in its entirety), and published in the scientific literature in an article by I. N. Weinberg, P. Y. Stepanov, S. T. Fricke, R. Probst, M. Urdaneta, D. Warnow, H. Sanders, S. C. Glidden, A. McMillan, P. M. Starewicz, and J. P. Reilly, entitled “INCREASING THE OSCILLATION FREQUENCY OF STRONG MAGNETIC FIELDS ABOVE 101 KHZ SIGNIFICANTLY RAISES PERIPHERAL NERVE EXCITATION THRESHOLDS,” in a May 2012 article in the journal Medical Physics, vol. 39, no. 5, pages 2578-83 (and incorporated by reference in its entirety).

By employing one or more magnetic gradient pulses with very short rise-times and/or fall-times (for example, less than 10 microseconds) as disclosed in U.S. Pat. No. 8,154,286, the magnitude of the magnetic gradients that can be applied to human nervous tissues without causing unwanted stimulation can be at least ten times higher than in the prior art (for example, 400 milliTeslas). Such high magnitudes would be similar to those previously obtained with permanent magnets for manipulating nanoparticles, as in the above-cited publication by Lubbe et al. Thus, the same coils used to produce propulsion can be used to create an image in the MRI scanner. As discussed above, the process of creating an image in an MRI scanner includes the alteration of rotational frequencies of materials in the body, through the application of pulsed magnetic gradients, typically by modifying the resonant frequencies of polarizable particles in a space-dependent manner. The use of propulsive coils to both propel MNPs and collect images with the MNI scanner implies that the pulsed magnetic fields used to propel the MNPs do not interfere with the process of collecting an image with the MRI scanner.

While certain illustrative embodiments have been described, it is evident that many alternatives, modifications, permutations and variations will become apparent to those skilled in the art in light of the foregoing description. While illustrated embodiments have been outlined above, it is evident that many alternatives, modifications and variations will be apparent to those skilled in the art. Accordingly, the various embodiments of the invention, as set forth above, are intended to be illustrative, not limiting. Various changes may be made without departing from the spirit and scope of the invention.

As a result, it will be apparent for those skilled in the art that the illustrative embodiments described are only examples and that various modifications can be made within the scope of the invention as defined in the appended claims.

What is claimed is:

1. An apparatus comprising:
at least one magnetizable particle introduced into a body part;
a plurality of magnets positioned in proximate relationship to the body part; and
a control unit,
wherein the control unit controls the plurality of magnets to expose the at least one magnetizable particle to at least one static magnetic field and at least one time-varying magnetic field to polarize and then move the at least one magnetizable particle.

2. The apparatus of claim 1, wherein the at least one time-varying magnetic field is opposite in alignment from the at least one static magnetic field and sufficiently strong compared to the at least one static magnetic field to produce a resultant total magnetic field oriented in the same direction as the at least one time-varying magnetic field but with a smaller magnetic intensity.

3. The apparatus of claim 1, wherein application of the at least one static magnetic field and the at least one time-varying magnetic field creates a focusing region for the at least one magnetizable particle.

4. The apparatus of claim 1, wherein the at least one time-varying magnetic field has a rise- or fall-time of less than 10 microseconds.

5. The apparatus of claim 1, wherein application of magnetic fields creates a nodal point.

6. The apparatus of claim 1, wherein application of the at least one static magnetic field and the at least one time-varying magnetic field creates a dispersal region for a magnetizable particle.

7. The apparatus of claim 1, wherein application of magnetic fields induces an anti-agglomeration behavior in a plurality of magnetizable particles including the at least one magnetizable particle.

8. The apparatus of claim 1, wherein at least one of the plurality of magnets is at least one electromagnetic coil that is cooled to increase magnetic field strength thereof.

9. The apparatus of claim 1, wherein at least one of the plurality of magnets is a magnet assembly is in a proximate relationship to the region of interest or encompasses the region of interest.

10. The apparatus of claim 1, further comprising a field shifting apparatus comprised of ferromagnetic mu-metal materials and/or superconducting materials which alters a location of the at least one static magnetic field and the at least one time-varying magnetic field.

11. The apparatus of claim 1, further comprising a metallic material which alters the shape and intensity of the at least one static magnetic field and the at least one time-varying magnetic field.

12. The apparatus of claim 1, wherein the control unit controls the plurality of magnets to expose the at least one magnetizable particle to the at least one static magnetic field and the at least one time-varying magnetic field to polarize and then move the at least one magnetizable particle a plurality of times.

13. A method comprising controlling a plurality of magnets positioned in proximate relationship to a body part using a control unit to expose at least one magnetizable particle to at least one static magnetic field and at least one time-varying magnetic field to polarize and then move the at least one magnetizable particle within the body part.

14. The method of claim 13, wherein the at least one time-varying magnetic field is opposite in alignment from the at least one static magnetic field and sufficiently strong compared to the at least one static magnetic field to produce a resultant total magnetic field oriented in the same direction as the at least one time-varying magnetic field but with a smaller magnetic intensity.

15. The method of claim 13, wherein the at least one time-varying magnetic field has a rise- or fall-time of less than 10 microseconds.

16. The method of claim 13, wherein application of magnetic fields creates a nodal point.

17. The method of claim 13, wherein application of the at least one static magnetic field and the at least one time-varying magnetic field creates a dispersal region for a magnetizable particle.

18. The method of claim 13, wherein application of further magnetic fields induces an anti-agglomeration behavior in a plurality of magnetizable particles including the at least one magnetizable particle.

19. The method of claim 13, wherein at least one of the plurality of magnets is at least one electromagnetic coil that is cooled to increase magnetic field strength thereof.

20. The method of claim 13, wherein at least one of the plurality of magnets is a magnet assembly that is in a proximate relationship to the region of interest or encompasses the region of interest.

21. The method of claim 13, further comprising using a field shifting apparatus comprised of ferromagnetic mu-metal materials and/or superconducting materials to alter a location of the at least one static magnetic field and the at least one time-varying magnetic field.

22. The method of claim 13, further comprising altering the shape and intensity of the at least one static magnetic field and the at least one time-varying magnetic field using a metallic material.

23. The method of claim 13, wherein the control unit controls the plurality of magnets to expose the at least one magnetizable particle to the at least one static magnetic field and the at least one time-varying magnetic field to polarize and then move the at least one magnetizable particle a plurality of times.
